(19) United States
(12) Patent Application Publication
(10) Pub. No.: US 2018/0356382 A1
(43) Pub. Date: Dec. 13, 2018

(54) PERSONAL GAS MONITOR DIAGNOSTIC SYSTEMS AND METHODS

(71) Applicant: Shanghai Eagle Safety Equipment Ltd., Shanghai (CN)

(72) Inventors: JiFei Chen, Shanghai (CN); Bo Li, Shanghai (CN); Lei Wang, Shanghai (CN); Zhiqiang Huo, Shanghai (CN)

(21) Appl. No.: 15/771,130

(22) PCT Filed: Oct. 26, 2015
(86) PCT No.: PCT/CN2015/092828

§ 371 Date: Aug. 24, 2018

(51) Int. Cl. G01N 33/00; G01N 21/3504

(52) U.S. Cl. CPC G01N 33/007; G01N 2201/0221; G01N 21/3504

(57) ABSTRACT

A personal gas monitor may include a housing configured to be worn or held by an individual, one or more components secured on or in the housing, and a diagnostic system within the housing and coupled to the one or more components. The diagnostic system tests the one or more components to determine whether or not component (s) are properly functioning.

### FIELD OF THE DISCLOSURE

Embodiments of the present disclosure generally relate to personal gas monitor diagnostic systems and methods, and, more particularly, to systems and methods for ensuring that a personal gas monitor is properly functioning.

### BACKGROUND OF THE DISCLOSURE

Gas sensors or monitors are used to measure concentrations of target gases within particular locations. Personal or portable gas sensors, detectors, or monitors (“personal gas monitors”) are used in various settings to detect hazardous gases. For example, fire and emergency personnel may wear or carry a personal gas monitor in hazardous areas to detect toxic gases, such as carbon monoxide. The personal gas monitor typically includes a gas-detecting medium that is operatively connected to an alarm or display. If the detected gas exceeds an unsafe threshold, an audible alarm may be emitted, and/or a visual alarm may be shown on a display.

As can be appreciated, when an individual uses a personal gas monitor, it is important that the gas monitor properly functions. For example, a faulty gas monitor may not be capable of alerting an individual to a presence of toxic gas. If the gas monitor malfunctions, the individual needs to be alerted of the malfunction so that the individual does not rely on a malfunctioning gas monitor.

### SUMMARY OF THE DISCLOSURE

Certain embodiments of the present disclosure provide a personal gas monitor that may include a housing configured to be worn or held by an individual, one or more components secured on or in the housing, and a diagnostic system within the housing and coupled to the one or more components. The diagnostic system periodically tests the component(s) to determine whether or not they are properly functioning. The components may include one or more of a gas sensor, an audio unit, one or more light-emitting members, a vibrator, or a battery, for example. The diagnostic system may switch between a diagnostic state in which the diagnostic system tests the one or more components, and a normal operating state in which the personal gas monitor senses a presence and level of at least one gas. The diagnostic system may include at least one control unit in communication with the one or more components, and at least one memory coupled to the control unit(s).

In at least one embodiment, the control unit(s) samples a sensor output signal from the gas sensor (such as an analog output signal from a fixed direct current 3.3 V source) and compares the sensor output signal to a sensor reference value stored in the at least one memory to determine whether or not the gas sensor is properly functioning.

In at least one embodiment, the control unit(s) samples an audio output signal from an audio unit (such as a buzzer or speaker) and compares the audio output signal to an audio reference value stored in the memory to determine whether or not the audio unit is properly functioning. The audio output signal may be sampled as a voltage signal. Optionally, the diagnostic system may include a microphone that senses the audio output signal as an analog audio output signal emitted by the audio unit.

In at least one embodiment, the control unit(s) samples a motion signal from a vibrator and compares the motion signal to a motion reference value stored in the memory to determine whether or not the vibrator is properly functioning. The motion signal may be sampled as a voltage signal. Optionally, the diagnostic system may include a motion sensor that senses the motion signal as an analog motion signal emitted by the vibrator. The motion sensor may include one or more of a microelectromechanical (MEMS) sensor, an accelerometer, a piezoelectric transducer, a potentiometer, one or more strain gauges, and/or the like.

In at least one embodiment, the control unit(s) samples a light signal from at least one light-emitting member and compares the light signal to a light reference value stored in the memory to determine whether or not the light-emitting member(s) is properly functioning. The light signal may be sampled as a voltage signal. Optionally, the diagnostic system may include a phototransistor that detects the light signal as an analog light signal emitted by the light-emitting member(s).

In at least one embodiment, the diagnostic system may include a comparator that compares at least one input voltage of the component(s) to at least one reference voltage.

Certain embodiments of the present disclosure provide a method of testing one or more components of a personal gas monitor that is configured to be worn or held by an individual. The method may include disposing a diagnostic system within a housing of the personal monitor, coupling the diagnostic system to one or more components secured in or on the housing, and testing the component(s) with the diagnostic system to determine whether or not the component(s) are properly functioning.

Certain embodiments of the present disclosure provide a personal gas monitor that may include a housing configured to be worn or held by an individual, a gas sensor secured on or in the housing, an audio unit secured on or in the housing, one or more light-emitting members secured on or in the housing, a vibrator secured on or in the housing, and a diagnostic system within the housing that is coupled to each of the gas sensor, the audio unit, the light-emitting member(s), and the vibrator. The diagnostic system periodically tests the audio unit, the light-emitting member(s), and the vibrator to determine whether or not the audio unit, the light-emitting member(s), and the vibrator are properly functioning. The diagnostic system switches between a diagnostic state in which the diagnostic system tests the audio unit, the light-emitting member(s), and the vibrator, and a normal operating state in which the personal gas monitor senses a presence and level of at least one gas.

### BRIEF DESCRIPTION OF THE DRAWINGS

FIG. 1 illustrates a front view of a personal gas monitor, according to an embodiment of the present disclosure.

FIG. 2 illustrates a simplified schematic diagram of a personal gas monitor, according to an embodiment of the present disclosure.

FIG. 3 illustrates a schematic diagram of a diagnostic system coupled to a gas sensor of a personal gas monitor, according to an embodiment of the present disclosure.

FIG. 4 illustrates a schematic diagram of a diagnostic system coupled to an audio unit of a personal gas monitor, according to an embodiment of the present disclosure.

FIG. 5 illustrates a schematic diagram of a diagnostic system coupled to an audio unit of a personal gas monitor, according to an embodiment of the present disclosure.

FIG. 6 illustrates a schematic diagram of a diagnostic system coupled to an audio unit of a personal gas monitor, according to an embodiment of the present disclosure.

FIG. 7 illustrates a schematic diagram of a diagnostic system coupled to a gas sensor of a personal gas monitor, according to an embodiment of the present disclosure.

FIG. 8 illustrates a schematic diagram of a diagnostic system coupled to a vibrator of a personal gas monitor, according to an embodiment of the present disclosure.

FIG. 9 illustrates a schematic diagram of a diagnostic system coupled to a light emitting diode of a personal gas monitor, according to an embodiment of the present disclosure.

FIG. 10 illustrates a schematic diagram of a diagnostic system coupled to components of a personal gas monitor, according to an embodiment of the present disclosure.

FIG. 11 illustrates a flow chart of a method of performing a diagnostic test on a gas sensor of a personal gas monitor, according to an embodiment of the present disclosure.

FIG. 12 illustrates a flow chart of a method of performing a diagnostic test on an audio unit of a personal gas monitor, according to an embodiment of the present disclosure.

FIG. 13 illustrates a flow chart of a method of performing a diagnostic test on a vibrator of a personal gas monitor, according to an embodiment of the present disclosure.

FIG. 14 illustrates a flow chart of a method of performing a diagnostic test on one or more light-emitting members of a personal gas monitor, according to an embodiment of the present disclosure.

FIG. 15 illustrates a flow chart of a method of performing a diagnostic test on one or more components of a personal gas monitor, according to an embodiment of the present disclosure.

### DETAILED DESCRIPTION OF THE DISCLOSURE

The foregoing summary, as well as the following detailed description of certain embodiments, will be better understood when read in conjunction with the appended drawings. As used herein, an element or step recited in the singular and preceded by the word “a” or “an” should be understood as not necessarily excluding the plural of the elements or steps. Further, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Certain embodiments of the present disclosure provide a personal gas monitor that may include a housing configured to be worn by or held by an individual, a gas sensor within the housing, and a control unit operatively connected to the gas sensor through a signal line. The control unit and the signal line are within the housing. The personal gas monitor may also include an audio unit (such as a speaker, buzzer, and/or the like), and a display. At least one diagnostic circuit is disposed within the housing. The diagnostic circuit may periodically test one or both of the signal line and the audio unit. Diagnostic firmware may be used to regularly check the functional components of the personal gas monitor. For example, the diagnostic firmware may be part of, or otherwise coupled to, the control unit. The diagnostic circuit may be coupled to the control unit. In at least one embodiment, the control unit may be part of the diagnostic circuit.

The control unit switches the personal gas monitor between gas sensing (normal operating) and diagnostic states. For example, the control unit may switch the personal gas monitor between the gas sensing and the diagnostic states at intervals of thirty seconds or less. The diagnostic circuit(s) may compare a reference voltage from the signal line to one or more stored values.

In at least one embodiment, an ultralow noise microphone may be disposed within the diagnostic circuit or otherwise used to detect if the output of the audio unit is operating in a normal fashion. If the audio unit is not operating in a normal fashion, an error alert may be shown on a display and/or broadcast through an audio signal.

In at least one embodiment, the diagnostic circuit may compare output voltages of one or more components of the personal gas monitor with one or more reference voltages to determine whether or not the components are properly functioning. If the output voltages are above or below particular thresholds, an error or fault condition may be present. For example, an output of a battery of the personal gas monitor may be compared to a reference voltage to determine whether or not the battery needs to be recharged.

In at least one embodiment, the personal gas monitor may include a phototransistor that is used to detect light output of one or more light emitting diodes LEDs) of the personal gas monitor. The detected light is compared to a stored reference light value to determine whether or not the LEDs are properly functioning.

In at least one embodiment, the personal gas monitor may include a vibrator coupled to a motion sensor, such as a microelectromechanical (MEMS) senor, an accelerometer, a piezoelectric transducer, and/or the like. The motion of the vibrator detected by the motion sensor may be compared to a stored reference value to determine whether or not the vibrator is properly functioning.

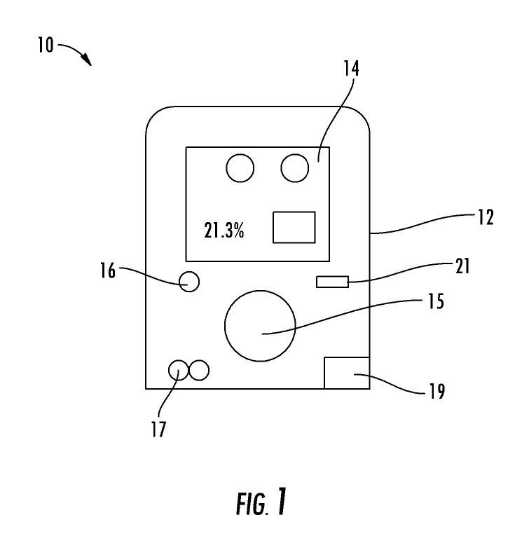

FIG. 1 illustrates a front view of a personal gas monitor 10, according to an embodiment of the present disclosure. The personal gas monitor 10 is an example of a monitor that is used to sense, detect, record, or otherwise monitor one or more attributes of an environment, location, area, or the like. For example, the personal gas monitor 10 is configured to detect a concentration, level, presence, or the like of one or more gases within a location surrounding the personal gas monitor 10.

The personal gas monitor 10 includes a housing 12 that is configured to be worn by an individual, such as on a belt, and/or held by the individual. The housing 12 contains or otherwise retains a gas sensor 15, one or more LEDs 17, a vibrator 19, and one or more processing circuits (not shown in FIG. 1), such as one or more diagnostic circuits. The personal gas monitor 10 may also include an audio unit 21 (such as a buzzer, speaker, or the like) configured to emit audible signals, such as alarms. The housing 12 may also include a display 14 (such as an LED, liquid crystal display, digital, or the like screen) configured to show information regarding a detected amount of one or more gases. A gas intake port 16 may be formed through the housing 12 and is in fluid communication with the gas sensor 15. The personal gas monitor 10 may be various shapes and sizes, other than shown. Alternatively, the personal gas monitor 10 may not include the display 14.

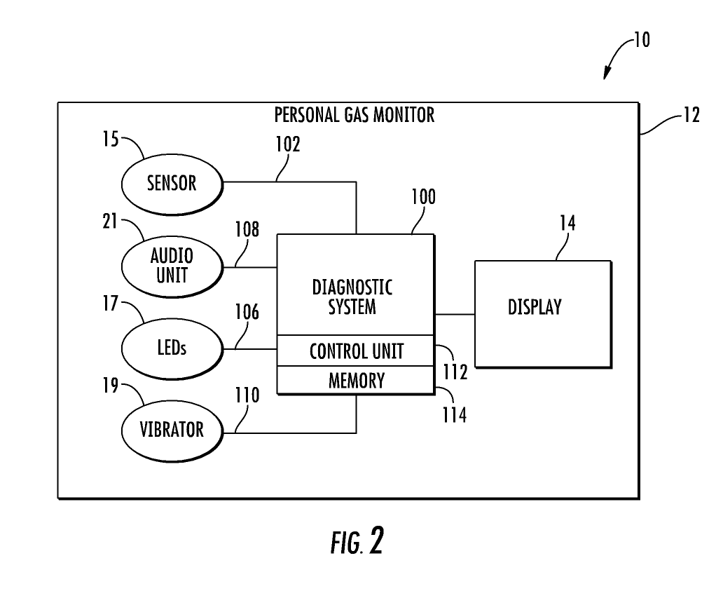

FIG. 2 illustrates a simplified schematic diagram of the personal gas monitor 10, according to an embodiment of the present disclosure. The personal gas monitor 10 includes an internal diagnostic system 100 coupled to the gas sensor 15, the LEDs 17, the vibrator 19, and the audio unit 21. As shown, a single diagnostic system 100 may be operatively coupled to the gas sensor 15, the LEDs 17, the vibrator 19, and the audio unit 21. Optionally, a separate and distinct diagnostic system 100 may be coupled to each of the gas sensor 15, the LEDs 17, the vibrator 19, and the audio unit 21. Alternatively, not all of the sensor 15, the LEDs 17, the vibrator 19, and the audio unit 21 may be coupled to a diagnostic system 100. Also, alternatively, the personal gas monitor 10 may not include all of the LEDs 17, the vibrator 19, and the audio unit 21. For example, the personal gas monitor 10 may not include the vibrator 19. In at least one other embodiment, the personal gas monitor 10 may not include the LEDs 17, or may include only one LED 17. In at least one other embodiment, the personal gas monitor 10 may not include the audio unit 21.

The sensor 15 may be coupled to the diagnostic system 100 through a signal line 102. The LEDs 17 may be coupled to the diagnostic system 100 through a signal line 104. The vibrator 19 may be coupled to the diagnostic system 100 through a signal line 106. The audio unit 21 may be coupled to the diagnostic system 100 through a signal line 108.

The diagnostic system 100 may include or be in communication with a control unit 112 and a memory 114. The diagnostic system 100 receives signals from one or more of the sensor 15, the LEDs 106, the vibrator 19, and the audio unit 21 to determine whether or not such components are properly functioning. In at least one embodiment, reference or threshold values may be stored in the memory 114. The control unit 112 may compare received test signals from the sensors 15, the LEDs 17, the vibrator 19, and/or the audio unit 21 with the stored reference or threshold values to determine whether or not the components are properly functioning.

The diagnostic system 100 may include one or more diagnostic circuits, or the like. For example, the diagnostic system 100 may include a sensor signal diagnostic circuit that detects the integrity of the sensor signal conveyed through the sensor signal line 102. The diagnostic system 100 may include an alarm control circuit that is configured to detect whether an alarm function (such as that of the audio unit 21) of the personal gas monitor 10 is properly functioning.

The diagnostic system 100 ensures that an individual uses a fully-functioning personal gas monitor 10. If the gas monitor is malfunctioning, the diagnostic system 100 may provide an audible and/or visual alert to the individual, such as on the display 14 and/or through the audio unit 21. Accordingly, the individual may then replace the malfunctioning gas monitor with a properly functioning gas monitor.

The diagnostic system 100 is configured to detect if the safety functions of the personal gas monitor 10 are in working condition or not. Accordingly, the diagnostic system 100 may allow the personal gas monitor 10 to be a safety integrity level (SIL) approved device. SIL may be defined as a relative level of risk-reduction provided by a safety function, and/or a specific target level of risk reduction. SIL measures performance required for a Safety Instrumented Function (SIF).

As used herein, the term “controller,” “control unit,” “central processing unit,” “CPU,” “computer,” or the like may include any processor-based or microprocessor-based system including systems using microcontrollers, reduced instruction set computers (RISC), application specific integrated circuits (ASICs), logic circuits, and any other circuit or processor capable of executing the functions described herein. Such are exemplary only, and are thus not intended to limit in any way the definition and/or meaning of such terms.

The control unit 112, for example, is configured to execute a set of instructions that are stored in one or more storage elements (such as one or more memories), in order to process data. For example, the control unit 112 may include or be coupled to one or more memories, such as the memory 114. The storage elements may also store data or other information as desired or needed. The storage element may be in the form of an information source or a physical memory element within a processing machine.

The set of instructions may include various commands that instruct the control unit 112 as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software may also include modular programming in the form of object-oriented programming. The processing of input data by the processing machine may be in response to user commands, or in response to results of previous processing, or in response to a request made by another processing machine.

The diagrams of embodiments herein may illustrate one or more control or processing units, such as the control unit 112. It is to be understood that the processing or control units may represent circuit modules that may be implemented as hardware with associated instructions (e.g., software stored on a tangible and non-transitory computer readable storage medium, such as a computer hard drive, ROM, RAM, or the like) that perform the operations described herein. The hardware may include state machine circuitry hardwired to perform the functions described herein. Optionally, the hardware may include electronic circuits that include and/or are connected to one or more logic-based devices, such as microprocessors, processors, controllers, or the like. Optionally, the control units may represent processing circuitry such as one or more of a field programmable gate array (FPGA), an application specific integrated circuit (ASIC), microprocessor(s), a quantum computing device, and/or the like. The circuits in various embodiments may be configured to execute one or more algorithms to perform functions described herein. The one or more algorithms may include aspects of embodiments disclosed herein, whether or not expressly identified in a flowchart or a method.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

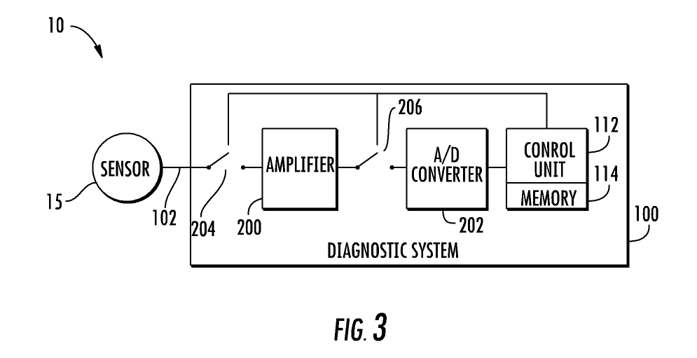

FIG. 3 illustrates a schematic diagram of the diagnostic system 100 coupled to the gas sensor 15 of the personal gas monitor 10, according to an embodiment of the present disclosure. The diagnostic system 100 may be or include a diagnostic circuit. The diagnostic system 100 may be contained within the housing 12 shown in FIGS. 1 and 2. The gas sensor 22 may be an electrochemical gas sensor, for example. The diagnostic system 100 may include an amplifier 200, an analog-to-digital (A/D) converter 202, the control unit 112, and the memory 114. Optionally, the control unit 112 and the memory 114 may not be part of the diagnostic system 100, but may be coupled to the diagnostic system 100. Switches 204 and 206 may be disposed within the diagnostic system 100 in order to switch between gas sensing and diagnostic functions. For example, during a gas sensing operation, the switches 204 and 206 may be operated by the control unit 112 to provide a gas sensing signal through a signal line, such as from the gas sensor 15 to the control unit 112.

The control unit 112 may periodically, such as every thirty seconds or less, switch the diagnostic system 100 to a diagnostic configuration in order to detect whether the gas sensor 15 is functioning as intended. The control unit 112 may detect a reference voltage signal from the gas sensor 15 or various other components to diagnose the various system components. For example, the control unit 112 may compare output voltages from the gas sensor 15 that are passed through the amplifier 200 and the A/D converter 202 with one or more stored values (which may be stored in the memory 114), for example, to determine whether the gas sensor 15 is properly functioning. As an example, if the output signals from the gas sensor 15 that are amplified by the amplifier 200 and converted from analog to digital by the A/D converter are below (or alternatively above) a sensor threshold value stored in the memory 114, the control unit 112 may determine that the gas sensor 15 is faulty, and may generate an alert signal (which may be emitted on an audio unit or shown on a display) that the gas sensor 15 is malfunctioning. In at least one embodiment, the control unit 112 may check each of the system components with respect to one or more stored reference values.

Alternatively, the diagnostic system may not include the amplifier 200 and the A/D converter 202. Instead, the sensor 15 may connect directly to the control unit 112 through the signal line 102. The control unit 112 may periodically switch the personal gas monitor 10 between gas sensing and diagnostic states, as noted above.

The diagnostic system 100 provides a diagnostic circuit for safety functions. The diagnostic system 100 may use one or more reference voltages with respect to feedback for the control unit 112. The control unit 112 may be configured to switch the diagnostic system 100 between normal operating and diagnostic states. The control unit 112 is configured to sample signals and compare them with stored data values to determine the integrity of the signal line from the gas sensor 15 to the control unit 112.

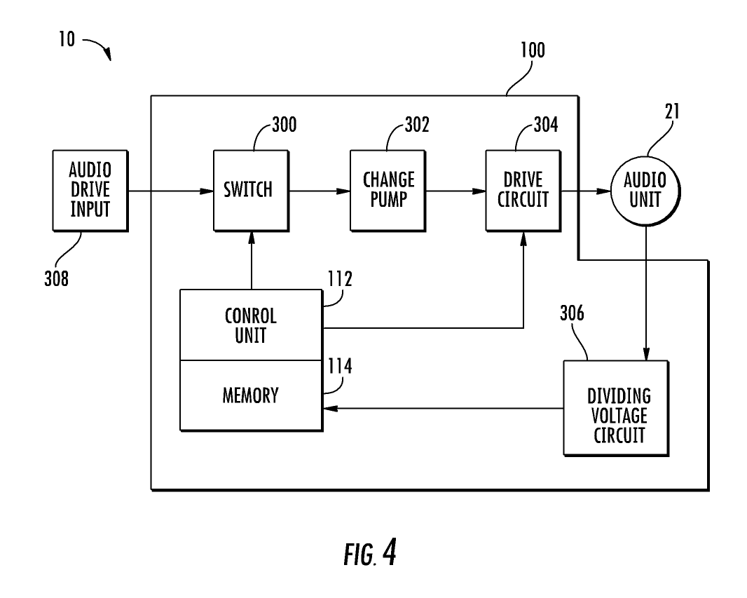

FIG. 4 illustrates a schematic diagram of the diagnostic system 100 coupled to the audio unit 21 of the personal gas monitor 10, according to an embodiment of the present disclosure. The diagnostic system 100 may include the control unit 112, the memory 114 (optionally, the control unit 112 and the memory 114 may be coupled to the diagnostic system 100), a switch 300, a charge pump 302, a drive circuit 304 coupled to the audio unit 21, and a dividing voltage circuit 306. The control unit 112 samples the drive voltage from the audio unit 21 to determine whether proper drive voltage is provided to the audio unit 21. The audio unit 40 may be a buzzer that is configured to buzz when the gas sensor detects a hazardous gas level. A faulty audio unit 21 may be unable to alert an individual to a hazardous gas level.

The diagnostic system 30 may be or include a buzzer voltage measurement circuit that is configured to detect a status of a safety function, such as an alarming function, of the portable gas monitor. The voltage dividing circuit 306 may sample the voltage provided to the audio unit 21. For example, an audio drive input 308, such as a voltage (for example, 4.5 V) is provided to the switch 300. The control unit 112 may operate the switch to transition the diagnostic system 100 into a diagnostic state. The audio drive input 308 is then passed to a charge pump 302 and a drive circuit 304, which may increase the audio drive input 308 to a driving voltage that is configured to activate the audio unit 21. The control unit 112 samples the driving voltage that is passed from the audio unit 21 through the dividing voltage circuit 306. The control unit 112 compares the sampled driving voltage to a stored reference voltage value within the memory 114. If the sampled voltage is above an audio threshold that may be defined by the stored reference voltage, the control unit 112 may deactivate the drive circuit 304 to disable the audio unit 21 (as the sampled voltage may indicate an excessive voltage that may damage the audio unit 21 or other components of the personal gas monitor 10). Conversely, if the sampled voltage is below a functional threshold, the control unit 112 may determine a fault condition in relation to the audio unit 21 and output an error signal, which may be shown on a display, for example. In short, the control unit 112 measures the sampled voltage and compares it with stored data (within the memory 114), such as a predetermined acceptable voltage level, to determine if the voltage provided to the audio unit 21 is at an acceptable level and/or within an acceptable range thereof.

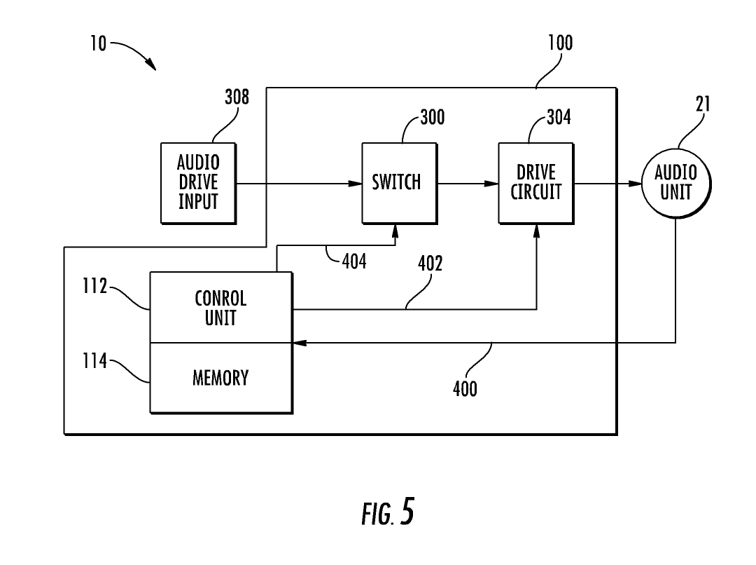

FIG. 5 illustrates a schematic diagram of the diagnostic system 100 coupled to the audio unit 21 of the personal gas monitor 10, according to an embodiment of the present disclosure. The embodiment of the diagnostic system 100 shown in FIG. 5 is similar to the embodiment shown in FIG. 4, except that the diagnostic system 100 may not include the charge pump 302 and the dividing voltage circuit 306. Instead, the audio drive input 308 is sent through the switch 300 and the drive circuit 304, which boosts the audio drive input to a driving voltage (such as 12 V). The control unit 112 then samples the driving voltage directly from the audio unit 21, and compares the sampled voltage to one or more reference voltages stored in the memory 114. As such, a diagnostic signal path 400 directly connects the audio unit 21 to the control unit 112.

The control unit 112 is also connected to the drive circuit 304 through a control path 402, which is configured to allow control signals to pass from the control unit 112 to the drive circuit 304. As noted above, if the control unit 112 determines that the sampled drive voltage exceeds a particular stored threshold, the control unit 112 may deactivate the drive circuit 304 so that the audio unit 21 may not be operated (in order to prevent damage to the audio unit 21 and/or other components of the personal gas monitor 10).

Further, the control unit 112 is connected to the switch through a switch path 404. The control unit 112 sends switching signals to the switch 300 through the switch path 404. The switch signals switch the diagnostic system 100 between a normal operating state and a diagnostic state in which the drive voltage supplied to the audio unit 21 is sampled to determine whether or not the audio unit is safely and properly functioning.

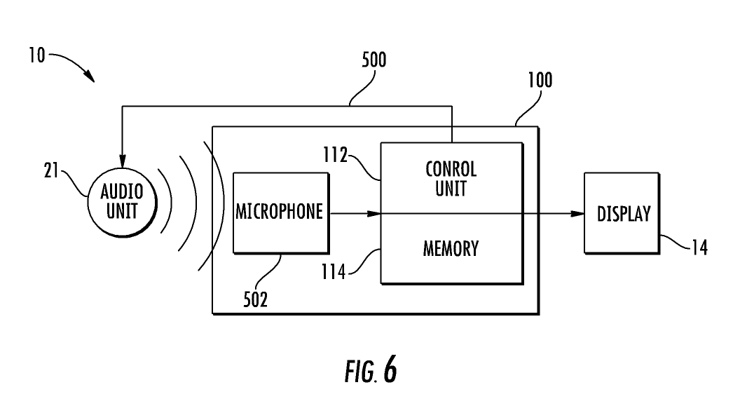

FIG. 6 illustrates a schematic diagram of the diagnostic system 100 coupled to the audio unit 21 of the personal gas monitor 10, according to an embodiment of the present disclosure. The control unit 100 may be coupled to the audio unit 21 through a drive path 500 that causes the audio unit 21 (such as a buzzer) to emit an audio signal. The diagnostic system 100 may include a microphone 502 that detects the audio signal, which is then relayed to the control unit 112 for comparison. For example, an A/D converter may convert the audio signal relayed from the microphone 502 into a digital signal that may be analyzed by the control unit 112. The control unit 112 then compares the received audio signal to a stored reference signal within the memory 114. If the sampled audio signal exceeds a particular threshold, the control unit 112 may disable the audio unit 21, as described above. Additionally, the control unit 112 may send an audio status signal to the display 14, which may display a current status of the audio unit 21 that is based on an analysis of the sampled audio signal by the control unit 112.

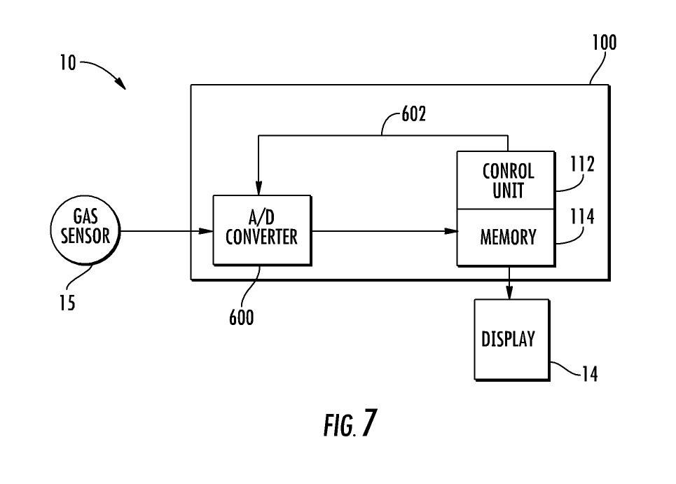

FIG. 7 illustrates a schematic diagram of the diagnostic system 100 coupled to the gas sensor 15 of the personal gas monitor 10, according to an embodiment of the present disclosure. In this embodiment, the diagnostic system 100 may include an A/D converter 600 that receives an analog sensor output signal from the gas sensor 15. The A/D converter 600 converts the analog sensor output signal to a digital output signal that is sent to the control unit 112. The control unit 112 may then compare the received digital output signal to a stored reference value within the memory 114 to determine whether or not the gas sensor 15 is active. The control unit 112 may then send a sensor status signal to the display 14, which may then display a status of the gas sensor 15 that is based on the sensor status signal.

In at least one embodiment, the control unit 112 may provide a logical signal (such as a “1”) to the A/D converter 600 through a signal path 602. The A/D converter 600 may convert the analog sensor output to a logical signal, which may then be sent to the control unit 112. If the logical signals match, the control unit 112 may determine that the gas sensor 15 is active. If, however, the logical signals do not match, the control unit 112 may determine that the gas sensor 15 is deactivated.

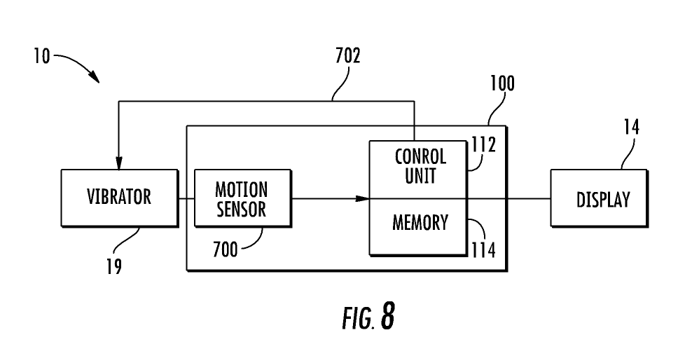

FIG. 8 illustrates a schematic diagram of the diagnostic system 100 coupled to the vibrator 19 of the personal gas monitor 10, according to an embodiment of the present disclosure. The diagnostic system 100 may include a motion sensor 700 that is coupled to the vibrator 19. The motion sensor 700 may be, for example, a MEMS sensor, an accelerometer, a piezoelectric transducer, a potentiometer, one or more strain gauges, and/or the like.

In the diagnostic state, the control unit 112 sends a vibration signal to the vibrator along a signal path 702. The vibration signal 702 is intended to cause the vibrator 19 to vibrate. As the vibration signal is sent, the motion sensor 700 senses any motion generated by the vibrator 19. The motion sensor 700 may be coupled to an A/D converter that converts the analog signal to a digital signal, which is then sent to the control unit 112. The control unit 112 compares the received digital vibration signal to a reference value stored in the memory 114 to determine whether or not the vibrator 19 is properly functioning. The control unit 112 may then send a vibrator status signal to the display 14, which may then show a corresponding status of the vibrator 19.

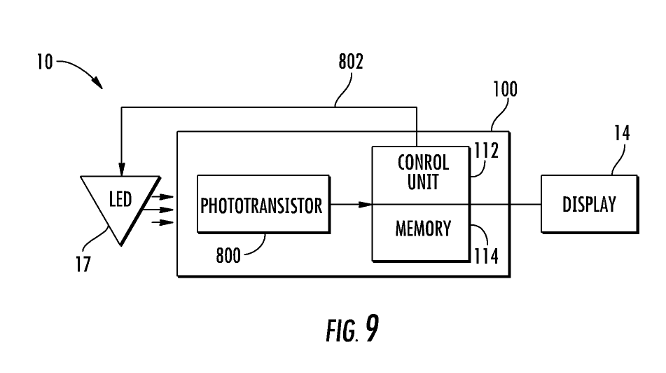

FIG. 9 illustrates a schematic diagram of the diagnostic system 100 coupled to one or more LEDs 17 of the personal gas monitor 10, according to an embodiment of the present disclosure. The diagnostic system 100 may include a phototransistor 800 that is exposed to the LEDs 17. The phototransistor 800 is configured to detect a light output from the LEDs 17.

In the diagnostic state, the control unit 112 sends a light-emitting signal to the LEDs 17 along a signal path 802. The light-emitting signal is intended to cause the LEDs 17 to emit light. As the light-emitting signal is sent, the phototransistor 800 detects light (if any) emitted by the LEDs 17. The phototransistor 800 may be coupled to an A/D converter that converts the analog signal to a digital signal, which is then sent to the control unit 112. The control unit 112 compares the received digital light signal to a reference value stored in the memory 114 to determine whether or not the LEDs 17 are properly functioning. The control unit 112 may then send a light status signal to the display 14, which may then show a corresponding status of the LEDs 17

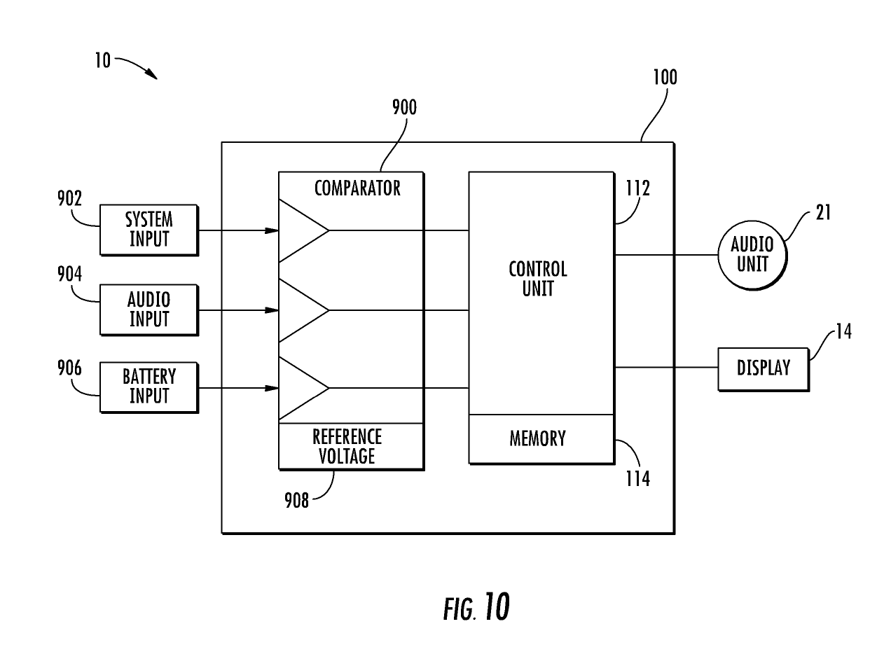

FIG. 10 illustrates a schematic diagram of the diagnostic system 100 coupled to components of the personal gas monitor 10, according to an embodiment of the present disclosure. The diagnostic system 100 may include a comparator 900 coupled to the control unit 112. The comparator 900 includes a plurality of inputs configured to receive various input voltages that are configured to drive various components of the personal gas monitor 10. For example, the comparator 900 may receive a system input 902 that is configured to drive the control unit 112 and and/or other electrical circuits within the personal gas monitor 10. For example, an acceptable system input 902 may be 3.3 V. The comparator 900 may also receive an audio input 904 that is configured to drive the audio unit 21. For example, an acceptable audio input 904 may be 4.2 V. The comparator 900 may also receive a battery input 906 from a power source (such as a battery). For example, an acceptable battery input 906 may be 3.7 V.

The comparator receives the system input 902, the audio input 904, and the battery input 906 and compares them to one or more reference voltages 908. If the inputs 902, 904, and 906 are within an acceptable range of the reference voltage(s) 908, the comparator 900 outputs corresponding logical states (such as “0” or “1”) to the control unit 112. The control unit 112 may then compare the logical states related to each of the system input 902, the audio input 904, and the battery input 906 and compare them with corresponding threshold logical states within the memory 114. If the logical states match, then the control unit 112 may determine that the components are properly functioning. If, however, the logical states are the opposite of the stored logical states, the control unit 112 may then determine that a fault or error condition exists, and may send appropriate alarm signals to the audio unit 21 and/or the display 14.

Referring to FIGS. 1-10, the diagnostic system 100 may be a single diagnostic system 100 coupled to all of the components described above. Optionally, separate and distinct diagnostic systems 100 may be coupled to separate and distinct components. For example, the personal gas monitor 10 may include the diagnostic system 100 shown in FIG. 4 and a separate and distinct diagnostic system, such as the diagnostic system 100 shown in FIG. 7. In at least one embodiment, a diagnostic system 100 may include a single control unit 112 coupled to the various components of the personal gas monitor. In at least one other embodiment, each component (such as the gas sensor 15, the LEDs 17, the vibrator 19, and the audio unit 21) may be coupled to a separate and distinct control unit 112.

The diagnostic testing functions described in FIGS. 1-10 may be conducted serially or in parallel, for example. For example, the diagnostic system 100 may be configured to perform diagnostic tests for all of the components of the personal gas monitor 10. Optionally, the diagnostic system 100 may be configured to perform diagnostic tests for less than all of the components of the personal gas monitor. In at least one embodiment, each of the diagnostic functions described may occur at the same time. In at least one other embodiment, less than all of the diagnostic functions may be utilized. For example, only one diagnostic function regarding an audio unit may be utilized.

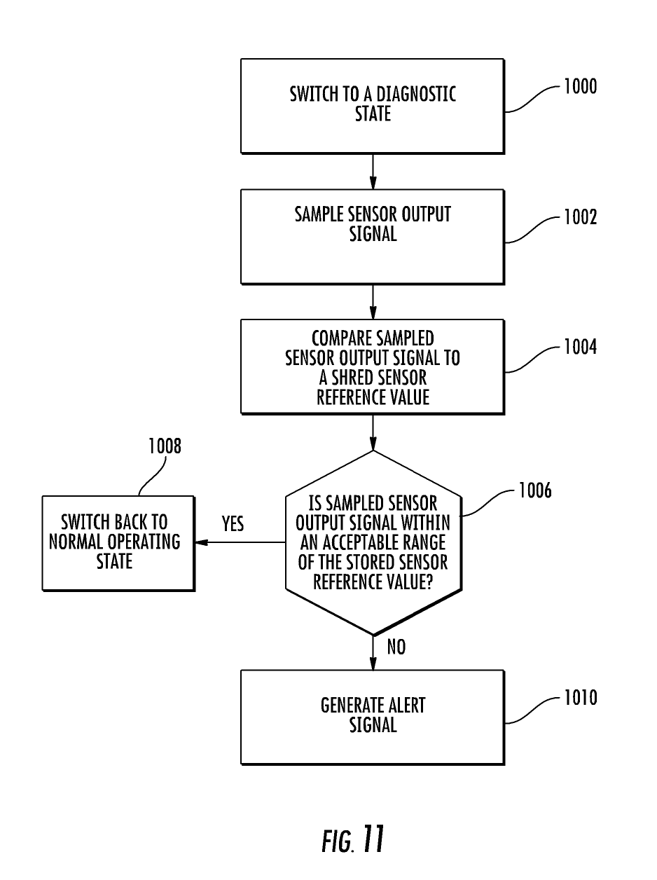

FIG. 11 illustrates a flow chart of a method of performing a diagnostic test on a gas sensor of a personal gas monitor, according to an embodiment of the present disclosure. One or more control units, such as the control unit 112, may be configured to operate according to the flow chart shown and described with respect to FIG. 11.

The method begins at 1000, in which the personal gas monitor is switched from a normal operating state to a diagnostic state. Then, at 1002, a sensor output signal (such as a sensor output voltage) is sampled from the sensor. At 1004, the sampled sensor output signal is compared to a stored sensor reference value. At 1006, it is determined if the sampled sensor output signal is within an acceptable range of the stored sensor reference value. For example, the sampled sensor output signal may be the same as the stored sensor reference value, or within a predetermined and predefined acceptable limit, such as within 5%. If the sampled sensor output signal is within an acceptable range of the stored sensor reference value, the method proceeds from 1006 to 1008, in which the personal gas monitor is switched back to the normal operating state. If, however, the sampled sensor output signal is not within the acceptable range of the stored sensor reference value, the method proceeds from 1006 to 1010, in which an alert signal is generated. The alert signal indicates that the gas sensor is not properly functioning, and may be shown on a display and/or emitted through an audio unit.

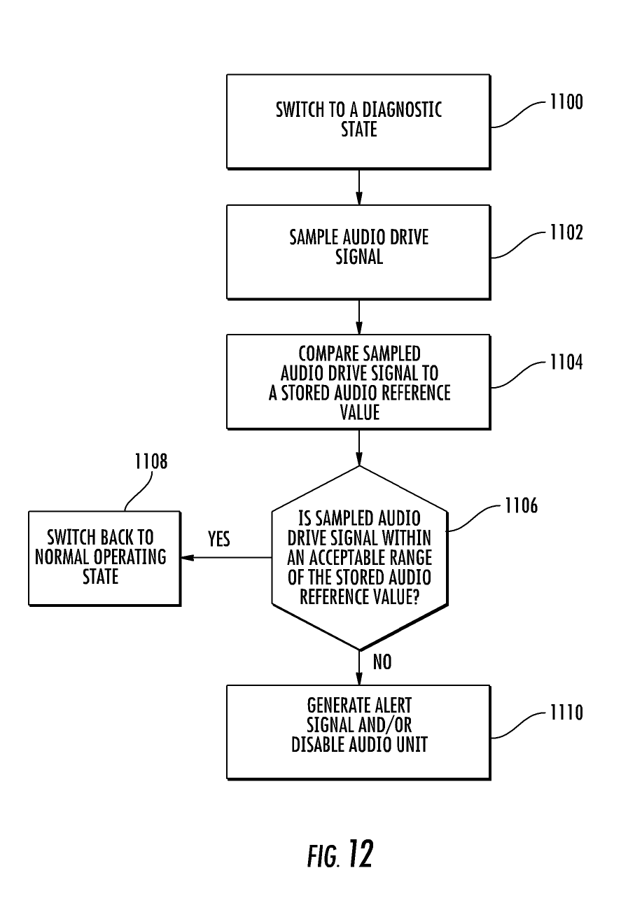

FIG. 12 illustrates a flow chart of a method of performing a diagnostic test on an audio unit of a personal gas monitor, according to an embodiment of the present disclosure. One or more control units, such as the control unit 112, may be configured to operate according to the flow chart shown and described with respect to FIG. 12.

The method begins at 1100, in which the personal gas monitor is switched from a normal operating state to a diagnostic state. Then, at 1102, an audio drive signal (such as signal configured to drive a buzzer) is sampled. For example, the audio drive signal may be or include a drive voltage sampled from the audio unit. In at least one other embodiment, the audio drive signal may be an analog signal picked up by a microphone and converted to a digital signal. At 1104, the sampled audio drive signal is compared to a stored audio reference value. At 1106, it is determined if the sampled audio drive signal is within an acceptable range of the stored audio reference value. For example, the sampled audio drive signal may be the same as the stored audio reference value, or within a predetermined and predefined acceptable limit, such as within 5%. If the sampled audio drive signal is within an acceptable range of the stored audio reference value, the method proceeds from 1106 to 1108, in which the personal gas monitor is switched back to the normal operating state. If, however, the sampled audio drive signal is not within the acceptable range of the stored audio reference value, the method proceeds from 1106 to 1110, in which an alert signal is generated and/or the audio unit is disabled. The alert signal indicates that the audio unit is not properly functioning, and may be shown on a display and/or emitted through an audio unit.

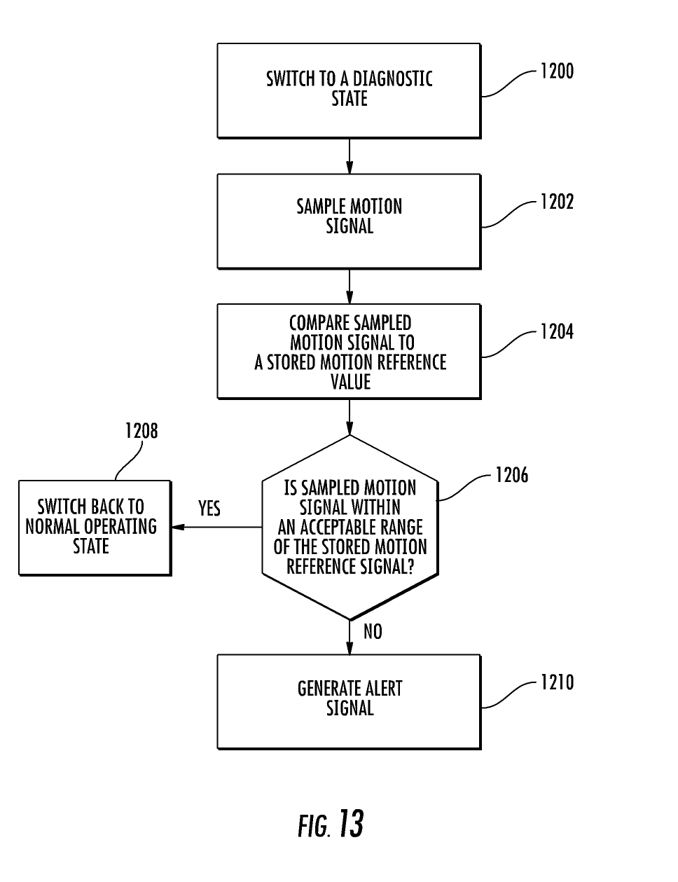

FIG. 13 illustrates a flow chart of a method of performing a diagnostic test on a vibrator of a personal gas monitor, according to an embodiment of the present disclosure. One or more control units, such as the control unit 112, may be configured to operate according to the flow chart shown and described with respect to FIG. 13.

The method begins at 1200, in which the personal gas monitor is switched from a normal operating state to a diagnostic state. Then, at 1202, a motion signal (such as signal configured to drive a vibrator) is sampled. For example, the motion signal may be or include a drive voltage sampled from the vibrator. In at least one other embodiment, the motion signal may be an analog signal picked up by a motion sensor and converted to a digital signal. At 1204, the sampled motion signal is compared to a stored motion reference value. At 1206, it is determined if the sampled motion signal is within an acceptable range of the stored motion reference value. For example, the sampled motion signal may be the same as the stored motion reference value, or within a predetermined and predefined acceptable limit, such as within 5%. If the sampled motion signal is within an acceptable range of the stored motion reference value, the method proceeds from 1206 to 1208, in which the personal gas monitor is switched back to the normal operating state. If, however, the sampled motion signal is not within the acceptable range of the stored motion reference value, the method proceeds from 1206 to 1210, in which an alert signal is generated and/or the vibrator is disabled. The alert signal indicates that the vibrator is not properly functioning, and may be shown on a display and/or emitted through an audio unit.

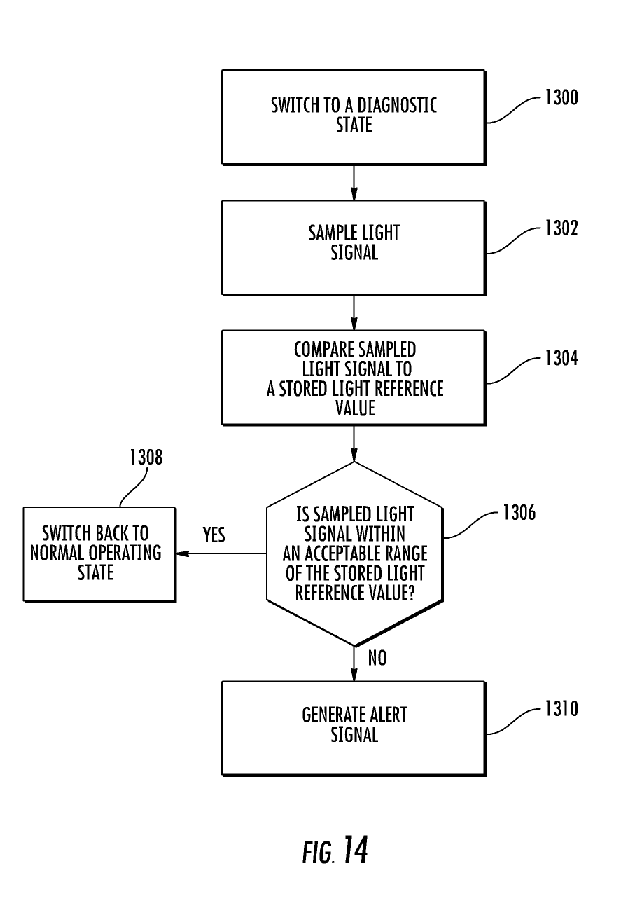

FIG. 14 illustrates a flow chart of a method of performing a diagnostic test on one or more light-emitting members of a personal gas monitor, according to an embodiment of the present disclosure. The light-emitting members may be or include one or more LEDs, one or more incandescent light bulbs, one or more fluorescent light bulbs, one or more infrared lights, one or more ultraviolet lights, and/or the like. One or more control units, such as the control unit 112, may be configured to operate according to the flow chart shown and described with respect to FIG. 14.

The method begins at 1300, in which the personal gas monitor is switched from a normal operating state to a diagnostic state. Then, at 1302, a light signal (such as signal configured to drive a light-emitting member) is sampled. For example, the light signal may be or include a voltage sampled from the light-emitting member(s). In at least one other embodiment, the light signal may be an analog signal detected by a phototransistor and converted to a digital signal. At 1304, the sampled light signal is compared to a stored light reference value. At 1306, it is determined if the sampled light signal is within an acceptable range of the stored light reference value. For example, the sampled light signal may be the same as the stored light reference value, or within a predetermined and predefined acceptable limit, such as within 5%. If the sampled light signal is within an acceptable range of the stored light reference value, the method proceeds from 1306 to 1308, in which the personal gas monitor is switched back to the normal operating state. If, however, the sampled light signal is not within the acceptable range of the stored motion reference value, the method proceeds from 1306 to 1310, in which an alert signal is generated and/or the light-emitting member(s) is disabled. The alert signal indicates that the light-emitting member(s) is not properly functioning, and may be shown on a display and/or emitted through an audio unit.

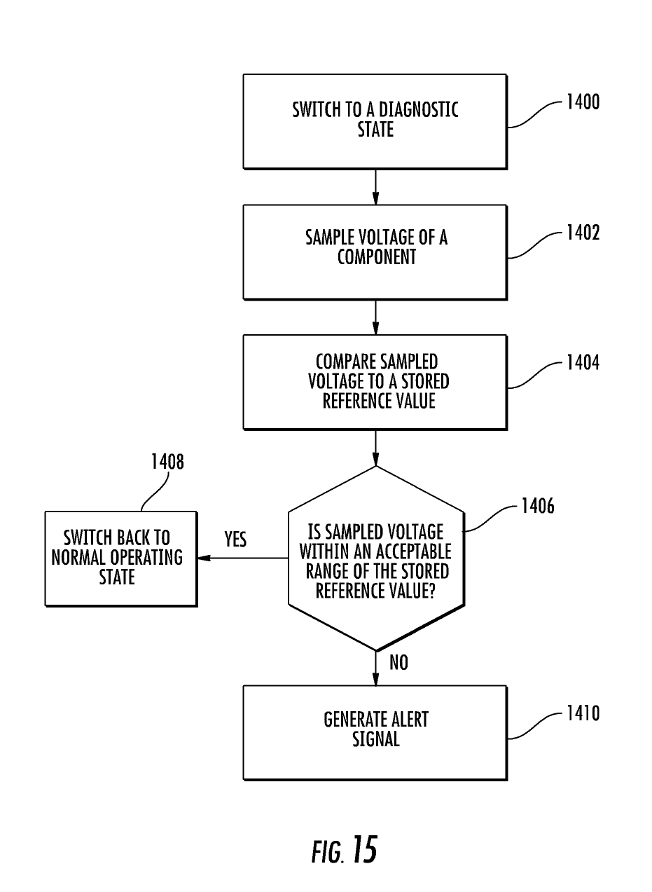

FIG. 15 illustrates a flow chart of a method of performing a diagnostic test on one or more components of a personal gas monitor, according to an embodiment of the present disclosure. The components may be or include an audio unit, one or more light-emitting members, a vibrator, a gas sensor, a battery, and/or the like. One or more control units, such as the control unit 112, may be configured to operate according to the flow chart shown and described with respect to FIG. 15.

The method begins at 1400, in which the personal gas monitor is switched from a normal operating state to a diagnostic state. Then, at 1402, a voltage of a component is sampled. At 1404, the sampled voltage is compared to a stored reference value. At 1406, it is determined if the sampled voltage is within an acceptable range of the stored reference value. For example, the sampled voltage may be the same as the stored reference value, or within a predetermined and predefined acceptable limit, such as within 5%. If the sampled voltage is within an acceptable range of the stored reference value, the method proceeds from 1406 to 1408, in which the personal gas monitor is switched back to the normal operating state. If, however, the sampled voltage is not within the acceptable range of the stored motion reference value, the method proceeds from 1406 to 1410, in which an alert signal is generated and/or the component is disabled. The alert signal indicates that the component is not properly functioning, and may be shown on a display and/or emitted through an audio unit.

Referring to FIGS. 11-15, one control unit 112 may be used to perform the various diagnostic tests. Optionally, more than one control unit 112 may be used to perform the various diagnostic tests. The diagnostic tests shown and described with respect to FIGS. 11-15 may be performed concurrently or at different times. Moreover, less than all of the diagnostic tests shown and described with respect to FIGS. 11-15 may be performed.

As explained above with respect to FIGS. 1-15, embodiments of the present disclosure provide systems and methods of performing diagnostic tests on one or more components of a personal gas monitor to ensure that the components properly function. As such, embodiments of the present disclosure provide systems and methods of alerting individuals of faulty personal gas monitors.

While various spatial and directional terms, such as top, bottom, lower, mid, lateral, horizontal, vertical, front and the like may be used to describe embodiments of the present disclosure, it is understood that such terms are merely used with respect to the orientations shown in the drawings. The orientations may be inverted, rotated, or otherwise changed, such that an upper portion is a lower portion, and vice versa, horizontal becomes vertical, and the like.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the disclosure without departing from their scope. While the dimensions and types of materials described herein are intended to define the parameters of the various embodiments of the disclosure, the embodiments are by no means limiting and are exemplary embodiments. Many other embodiments will be apparent to those of skill in the art upon reviewing the above description. The scope of the various embodiments of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various embodiments of the disclosure, including the best mode, and also to enable persons skilled in the art to practice the various embodiments of the disclosure, including making and using any devices or systems and performing any incorporated methods. The patentable scope of the various embodiments of the disclosure is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

1. A personal gas monitor, comprising:
a housing configured to be worn or held by an individual;
one or more components secured on or in the housing; and
a diagnostic system within the housing and coupled to the one or more components, wherein the diagnostic system tests the one or more components to determine whether or not the one or more components are properly functioning.

2. The personal gas monitor of claim 1, wherein the one or more components comprise one or more of a gas sensor, an audio unit, one or more light-emitting members, a vibrator, or a battery.

3. The personal gas monitor of claim 1, wherein the diagnostic system switches between a diagnostic state in which the diagnostic system tests the one or more components, and a normal operating state in which the personal gas monitor senses a presence and level of at least one gas.

4. The personal gas monitor of claim 1, wherein the diagnostic system comprises:
at least one control unit in communication with the one or more components; and
at least one memory coupled to the at least one control unit.

5. The personal gas monitor of claim 4, wherein the one or more components comprise a gas sensor, and wherein the at least one control unit samples a sensor output signal from the gas sensor and compares the sensor output signal to a sensor reference value stored in the at least one memory to determine whether or not the gas sensor is properly functioning.

6. The personal gas monitor of claim 4, wherein the one or more components comprise an audio unit, and wherein the at least one control unit samples an audio output signal from the audio unit and compares the audio output signal to an audio reference value stored in the at least one memory to determine whether or not the audio unit is properly functioning.

7. The personal gas monitor of claim 6, wherein the audio output signal is sampled as a voltage signal.

8. The personal gas monitor of claim 6, wherein the diagnostic system comprises a microphone that senses the audio output signal as an analog audio output signal emitted by the audio unit.

9. The personal gas monitor of claim 4, wherein the one or more components comprise a vibrator, and wherein the at least one control unit samples a motion signal from the vibrator and compares the motion signal to a motion reference value stored in the at least one memory to determine whether or not the vibrator is properly functioning.

10. The personal gas monitor of claim 7, wherein the motion signal is sampled as a voltage signal.

11. The personal gas monitor of claim 7, wherein the diagnostic system comprises a motion sensor that senses the motion signal as an analog motion signal emitted by the vibrator.

12. The personal gas monitor of claim 11, wherein the motion sensor comprises one or more of a microelectromechanical (MEMS) sensor, an accelerometer, a piezoelectric transducer, a potentiometer, or one or more strain gauges.

13. The personal gas monitor of claim 4, wherein the one or more components comprise at least one light-emitting member, and wherein the at least one control unit samples a light signal from the at least one light-emitting member and compares the light signal to a light reference value stored in the at least one memory to determine whether or not the at least one light-emitting member is properly functioning.

14. The personal gas monitor of claim 13, wherein the light signal is sampled as a voltage signal.

15. The personal gas monitor of claim 13, wherein the diagnostic system comprises a phototransistor that detects the light signal as an analog light signal emitted by the at least one light-emitting member.

16. The personal gas monitor of claim 1, wherein the diagnostic system comprises a comparator that compares at least one input voltage of the one or more components to at least one reference voltage.

17. A method of testing one or more components of a personal gas monitor that is configured to be worn or held by an individual, the method comprising:
disposing a diagnostic system within a housing of the personal monitor;
coupling the diagnostic system to one or more components secured in or on the housing; and
testing the one or more components with the diagnostic system to determine whether or not the one or more components are properly functioning.

18-24. (canceled)

25. A personal gas monitor, comprising:
a housing configured to be worn or held by an individual;
a gas sensor secured on or in the housing;
an audio unit secured on or in the housing;
one or more light-emitting members secured on or in the housing;
a vibrator secured on or in the housing; and
a diagnostic system within the housing and coupled to each of the gas sensor, the audio unit, the one or more light-emitting members, and the vibrator, wherein the diagnostic system periodically tests the audio unit, the one or more light-emitting members, and the vibrator to determine whether or not the audio unit, the one or more light-emitting members, and the vibrator are properly functioning, wherein the diagnostic system switches between a diagnostic state in which the diagnostic system tests the audio unit, the one or more light-emitting members, and the vibrator, and a normal operating state in which the personal gas monitor senses a presence and level of at least one gas.

26-30. (canceled)
